(19) United States
(12) Patent Application Publication
(10) Pub. No.: US 2026/0083912 A1
(43) Pub. Date: Mar. 26, 2026

(54) ADJUSTMENT OF MEDICAMENT DELIVERY BY A MEDICAMENT DELIVERY DEVICE BASED ON MENSTRUAL CYCLE PHASE

(71) Applicant: INSULET CORPORATION, Acton, MA (US)

(72) Inventor: Rangarajan NARAYANASWAMI, Weston, MA (US)

(21) Appl. No.: 19/405,974

(22) Filed: Dec. 2, 2025

Related U.S. Application Data
(63) Continuation of application No. 17/848,675, filed on Jun. 24, 2022, now Pat. No. 12,514,980.
(60) Provisional application No. 63/216,814, filed on Jun. 30, 2021.

(51) Int. Cl. A61M 5/172; G16H 20/17; G16H 40/67

(52) U.S. Cl. CPC A61M 5/1723; G16H 20/17; G16H 40/67; A61M 2205/3303; A61M 2205/50; A61M 2230/06; A61M 2230/201; A61M 2230/50; A61M 2230/63; A61M 2230/65

(57) ABSTRACT

Exemplary embodiments account for differing needs of a user over the menstrual cycle of the user to better control the blood glucose concentration of the user. The exemplary embodiments may be realized in control systems for medicament delivery devices that deliver medicaments, such as medicaments that regulate blood glucose concentration levels. Examples of such medicaments that regulate blood glucose concentration levels include insulin, glucagon, and glucagon peptide-1 (GLP-1) agonists. The exemplary embodiments are able to better tailor the dosages of the medicament delivered to the user with the medicament delivery device to reduce the risk of hyperglycemia and hypoglycemia and help reduce blood glucose concentration excursions.

### RELATED APPLICATIONS

This application is a continuation of U.S. patent application Ser. No. 17/848,675, filed Jun. 24, 2022, which claims the benefit of U.S. Provisional Ser. No. 63/216,814, filed Jun. 30, 2021, the contents of which are incorporated herein by reference in their entirety.

### BACKGROUND

A conventional medicament delivery device may deliver medicament at basal dosages and/or bolus dosages. The basal dosages are delivered on an-ongoing basis and aim to address a substantial portion of the need for the medicament on an on-going basis. The bolus dosages are delivered when requested by a user or when the control system for the medicament delivery device concludes based on information regarding the user that there is a need to deliver the bolus dosage. In some instances, the basal dosages are delivered automatically by the medicament delivery device to the user. The control system is generally responsible for determining the timing and the dosages for such medicament deliveries.

One example of a conventional medicament delivery device is an insulin delivery device, such as an insulin pump or patch. The conventional insulin delivery device may calculate a basal dosage based on total daily insulin (TDI). TDI represents the aggregate amount of insulin needed by the user for a day. TDI is typically calculated based on the weight of the user. A commonly used formula is that TDI equals user weight in pounds divided by 4. Thus, the TDI for a 200-pound man is 200 divided by 4 or 50 units of insulin. The basal dosage is conventionally determined to be one half of TDI.

Insulin boluses are typically delivered in response to meals by the user. The meals will increase the user's blood glucose concentration. The magnitude of the increase in blood glucose concentration is related to the quantity of carbohydrates ingested. Thus, a conventional insulin delivery device may determine what dosage of insulin will compensate for the quantity of carbohydrates ingested. In order to determine the amount of insulin needed and hence the bolus dosage, the conventional insulin delivery system may multiply the carbohydrates ingested by the insulin to carbohydrates ratio (ICR). The ICR conventionally may be set as a value typically selected from the range of 4 to 50. For example, a value of 4 for the ICR implies that 1 unit of insulin is to be delivered for every 4 grams of carbohydrates ingested. The conventional insulin delivery system may also look at the current blood glucose concentration and the insulin on board (IOB) for the user, which represents the quantity of insulin delivered to user that still has insulin action remaining.

### SUMMARY

In accordance with an inventive aspect of an embodiment, a method is performed by a processor in an electronic device. The method includes receiving information regarding a menstrual cycle of a user and based on the received information regarding a menstrual cycle of a user, adjusting a medicament dosage to be delivered by an automated medicament delivery device.

The received information may be a current phase of a menstrual cycle of the user. The received information may be received from a machine learning model. The received information may be information from which a current phase of the menstrual cycle is determined. The received information may include information from one or more sensors secured to the user. The medicament may be one of insulin, glucagon or a glucagon peptide-1 (GLP-1) agonist. The adjusting may include adjusting an insulin dosage to be delivered by the automated medicament delivery device based on an insulin sensitivity of the user for a current phase of the menstrual cycle of the user.

In accordance with another inventive feature of an embodiment, a method is performed by a processor in an electronic device. The method includes receiving input from a sensor. Based at least in part on the received input, a phase of a menstrual cycle of a user is determined with the processor. The method also includes determining insulin sensitivity of the user with the processor based on the determined phase of the menstrual cycle and adjusting the insulin delivered by a delivery device based on the determined insulin sensitivity.

The sensor may sense skin temperature, heart rate, skin conductance, or activity level. The method may further include receiving additional inputs from multiple sensors and using the additional inputs in the determining of the phase of the menstrual cycle of the user. The additional inputs from the multiple sensors may include a blood glucose concentration value for the user from one of the sensors that is a glucose monitor. The adjusting may adjust a size of a dosage of basal insulin to be delivered by the delivery device. The adjusting may adjust a size of dosage of an insulin bolus to be delivered by the delivery device.

In accordance with an additional aspect of an embodiment, a method is performed by a processor in an electronic device. Per the method, patterns of medicament sensitivity of a user based on a phase of a menstrual cycle of a user are learned by a machine learning model executing on the processor. Delivery of medicament to the user are adjusted by the processor based on the learned patterns to be delivered by a medicament delivery device to the user.

The method may further include receiving input from at least one sensor that senses information regarding the user and processing the input to determine a current phase of the menstrual cycle of the user. The input may include heart rate and skin temperature. The input may include blood glucose concentration and an indication of activity level. The learning may include training on a data set derived from women other than the user and subsequent to the training on the data set, training on data from the user to customize the machine learning model to the user. The medicament may be one of insulin, glucagon or a glucagon peptide-1 (GLP-1) agonist, for example.

### BRIEF DESCRIPTION OF THE DRAWINGS

FIG. 1A depicts an illustrative medicament delivery system that is suitable for delivering a medicament to a user in accordance with exemplary embodiments.

FIG. 1B shows a block diagram of examples of some sensors that may be used in exemplary embodiments.

FIG. 1C depicts one exemplary configuration of the sensors relative to the medicament delivery device.

FIG. 2 depicts a breakdown of the phases of an idealized menstrual cycle.

FIG. 3 depicts a flowchart of basic steps that may be performed in exemplary embodiments to compensate for changing insulin needs over the course of a menstrual cycle of a user.

FIG. 4A depicts a flowchart of illustrative steps that may be performed in exemplary embodiments to determine a current phase of a menstrual cycle of a user based on user-provided information.

FIG. 4B depicts a flowchart of illustrative steps that may be performed in exemplary embodiments to determine a current phase of a menstrual cycle of a user using a model.

FIG. 5 depicts a block diagram of the data flow for the machine learning model in exemplary embodiments.

FIG. 6 depicts a diagram showing some of the inputs that may be used by the machine learning model in exemplary embodiments.

FIG. 7 depicts a flowchart of illustrative steps that may be performed to determine in the current phase of the menstrual cycle of the user is the follicular phase.

FIG. 8 depicts a flowchart of illustrative steps that may be performed to determine in the current phase of the menstrual cycle of the user is the luteal phase.

FIG. 9A depicts a flowchart of illustrative steps that may be performed by exemplary embodiments to perform validation of the current phase of the menstrual cycle of the user based on the phase determined for the previous day.

FIG. 9B depicts a flowchart of illustrative steps that may be performed by exemplary embodiments to perform validation of current phase of the menstrual cycle of the user based on blood glucose concentration values.

FIG. 9C depicts a flowchart of illustrative steps that may be performed by exemplary embodiments to perform validation of current phase of the menstrual cycle of the user based on user-provided information.

FIG. 10 depicts a diagram showing the inputs and output of the basal adaptivity mechanism of the exemplary embodiments.

FIG. 11A depicts a flowchart of illustrative steps that may be performed in exemplary embodiments for follicular phase adjustments.

FIG. 11B depicts a flowchart of illustrative steps that may be performed in exemplary embodiments for luteal phase adjustments.

FIG. 12 depicts a diagram showing illustrative inputs and outputs for a bolus adaptivity mechanism.

FIG. 13 depicts a flowchart of illustrative steps that may be performed in exemplary embodiments to generate an adapted insulin bolus dosage based on current phase of the menstrual cycle of the user.

FIGS. 14A-14D depict illustrative user interfaces.

FIG. 15 depicts an illustrative user interface for soliciting information from a user regarding the user's menstrual cycle.

### DETAILED DESCRIPTION

The true medicament dosage needs of a user may vary from user to user. Moreover, the true medicament needs of a given user may vary over ranges of days or weeks and may vary even during a single day. Unfortunately, conventional medicament delivery devices do not account for many of these variations in medicament dosage needs. For example, a user's medicament dosage needs may vary based on the menstrual cycle of the user. One instance of this variation is that insulin sensitivity of a user may vary over the course of the menstrual cycle. Conventional insulin delivery devices do not account for this variation in insulin sensitivity due to the menstrual cycle phase of the user. This can be problematic in that the insulin needs of the user change over the course of the menstrual cycle of the user, but the conventional insulin delivery devices do not adjust for the change. As a result, the user may be at greater risk of hyperglycemia or hypoglycemia.

Exemplary embodiments described herein account for the differing needs of the user over the menstrual cycle of the user to better control the blood glucose concentration of the user. The exemplary embodiments may be realized in control systems for medicament delivery devices that deliver medicaments, such as medicaments that regulate blood glucose concentration levels. Examples of such medicaments that regulate blood glucose concentration levels include insulin, glucagon, and glucagon peptide-1 (GLP-1) agonists. The exemplary embodiments are able to better tailor the dosages of the medicament delivered to the user with the medicament delivery device to reduce the risk of hyperglycemia and hypoglycemia and help reduce blood glucose concentration excursions.

Some exemplary embodiments may use sensors, such as sensors of heart rate, skin temperature, skin conductance, and blood glucose concentration. The sensed values obtained from these sensors may be used to automatically determine a current phase of a menstrual cycle of the user. Once the current phase of the menstrual cycle is known the basal dosages and/or the bolus dosages automatically may be adjusted to account for changes associated with the phase. The adjustments may be made over time as new phases of the menstrual cycle are reached.

The exemplary embodiments may use a machine learning system to identify the phase of the menstrual cycle of the user from values obtained from the sensors. The machine learning system may learn the normal duration of each phase of the menstrual cycle as well as the normal length of a menstrual cycle for the user. The machine learning system may also learn how medicament sensitivity (such as insulin sensitivity) of the user varies over the course of the menstrual cycle for the user. In particular, the patterns of variance in magnitude and time may be learned.

In some exemplary embodiments, the machine learning system may rely upon a machine learning model that may utilize logistic regressors, random forests, deep learning networks, etc. The machine learning model may be trained on a data set derived from a large population of women and then may be customized to the user. Alternatively, the machine learning model may be trained solely on data derived from the user. The trained machine learning model may be used to identify the current phase of the menstrual cycle of the user.

In some exemplary embodiments no machine learning system is used. Instead, conventional logic may be used to identify the current phase of the menstrual cycle of the user. In other exemplary embodiments, a user identifies when the user's menstrual cycle began, and may also identify how long her menstrual cycle typically lasts, and that information from the user is used by the control system to adjust the medicament dosages.

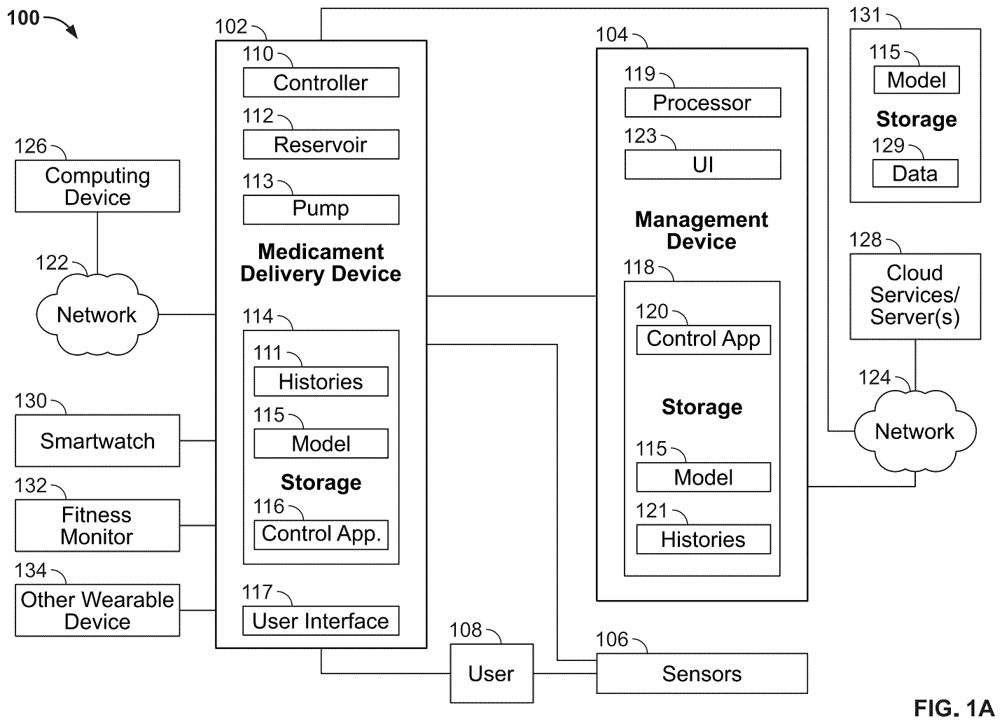

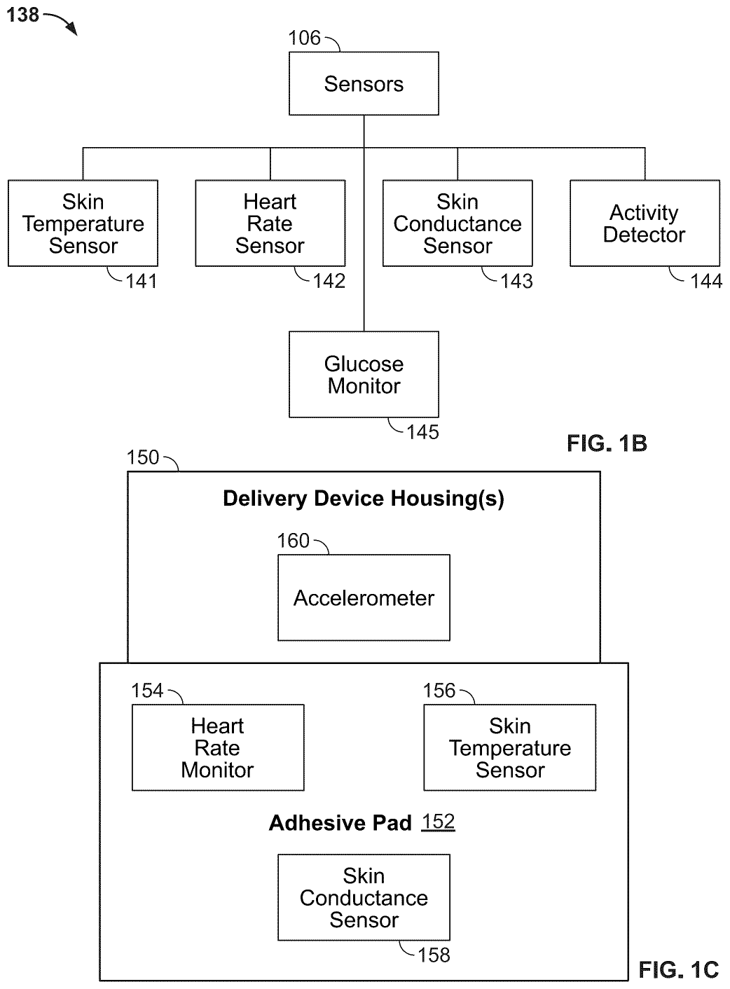

FIG. 1A depicts an illustrative medicament delivery system 100 that is suitable for delivering a medicament to a user 108 in accordance with exemplary embodiments. The medicament delivery system 100 includes a medicament delivery device 102. The medicament delivery device 102 may be a wearable device that is worn on the body of the user 108 or carried by the user and having an infusion site. The medicament delivery device 102 may be directly coupled to a user (e.g., directly attached to a body part and/or skin of the user 108 via an adhesive or the like) or carried by the user (e.g., on a belt or in a pocket) with tubing connecting the medicament delivery device 102 to an infusion site where the medicament is injected. In a preferred embodiment, a surface of the medicament delivery device 102 may include an adhesive to facilitate attachment to the user 108.

The medicament delivery device 102 may include a controller 110. The controller 110 may be implemented in hardware, software, or any combination thereof. The controller 110 may, for example, include a microprocessor, a logic circuit, a field programmable gate array (FPGA), an application specific integrated circuit (ASIC) or a microcontroller coupled to a memory. The controller 110 may maintain a date and time as well as other functions (e.g., calculations or the like). The controller 110 may be operable to execute a control application 116 encoded in computer programming instructions stored in the storage 114 that enables the controller 110 to direct operation of the medicament delivery device 102. The controller may in some exemplary embodiments execute a model 115, such as machine learning model to perform functionality as detailed below. In some embodiments, the model 115 is responsible for determining a current phase of the menstrual cycle of the user 108 and also learning patterns of key metrics, like heart rate, heart rate variability, skin temperature, and skin conductance over phases of the menstrual cycle of the user. The model 115 may also learn the average length of the phases of the menstrual cycle of the user and the average variation in insulin sensitivity over phases of the menstrual cycle of the user. The model 115 may be used to identify a current phase of the menstrual cycle of the user and to provide pattern information that is used to adapt basal dosages and bolus dosages of the medicament, like insulin. The model 115 may be realized in software.

The control application 116 may control delivery of a medicament to the user 108 per a control approach like that described herein. The storage 114 may hold histories 111 for a user, such as a history of basal deliveries, a history of bolus deliveries, and/or other histories, such as a meal event history, exercise event history and/or the like. In addition, the controller 110 may be operable to receive data or information. The storage 114 may include both primary memory and secondary memory. The storage may include random access memory (RAM), read only memory (ROM), optical storage, magnetic storage, removable storage media, solid state storage or the like.

The medicament delivery device 102 may include one or more housings for housing its various components including a pump 113, a power source, and a reservoir 112 for storing a medicament for delivery to the user 108 as warranted. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include tubing to a separate infusion site.

There may be one or more communications links with one or more devices physically separated from the medicament delivery device 102 including, for example, a management device 104 of the user and/or a caregiver of the user and/or a sensor 106. The communication links may include any wired or wireless communication links operating according to any known communications protocol or standard, such as Bluetooth®, Wi-Fi, a near-field communication standard, a cellular standard, or any other wireless protocol. The medicament delivery device 102 may also include a user interface 117, such as an integrated display device for displaying information to the user 108 and in some embodiments, receiving information from the user 108. The user interface 117 may include a touchscreen and/or one or more input devices, such as buttons, knobs, or a keyboard.

The medicament delivery device 102 may interface with a network 122. The network 122 may include a local area network (LAN), a wide area network (WAN) or a combination therein. A computing device 126 may be interfaced with the network, and the computing device may communicate with the medicament delivery device 102.

The medicament delivery system 100 may include sensors 106 for sensing the levels of one or more analytes. The sensors 106 may be coupled to the user 108 by, for example, adhesive or the like and may provide information or data on one or more medical conditions and/or physical attributes of the user 108. The sensors 106 may be physically separate from the medicament delivery device 102 or may be an integrated component thereof.

The medicament delivery system 100 may or may not also include a management device 104. In some embodiments, a management device is not needed as medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as processor, a micro-controller, or the like. The management device 104 may be used to program or adjust operation of the medicament delivery device 102 and/or the sensors 106. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's blood glucose levels and to control the delivery of the medicament to the user 108. The model 115 may run on the processor 119 of the management device 104 in some embodiments. The medicament delivery device 102 may provide data from the sensors 106 and other data to the management device 104. The data may be stored in the storage 118. The processor 119 may also be operable to execute programming code stored in the storage 118. For example, the storage may be operable to store one or more control applications 120 for execution by the processor 119. The one or more control applications 120 may be responsible for controlling the medicament delivery device 102, such as by controlling the AID delivery of insulin to the user 108. The storage 118 may store the one or more control applications 120, histories 121 like those described above for the medicament delivery device 102, and other data and/or programs.

The management device 104 may include a user interface (UI) 123 for communicating with the user 108. The user interface 123 may include a display, such as a touchscreen, for displaying information. The touchscreen may also be used to receive input when it is a touch screen. The user interface 123 may also include input elements, such as a keyboard, button, knobs, or the like.

The management device 104 may interface with a network 124, such as a LAN or WAN or combination of such networks. The management device 104 may communicate over network 124 with one or more servers or cloud services 128. In some exemplary embodiment, the model 115 and the data used by the model may be stored on the storage 131 for the cloud services/server(s) 128. The computational needs and the storage needs of the model 115 may be large, and the cloud services/server(s) 128 may be a suitable match for those needs. In such instances, the data, such as sensor values, may be sent for storage and processing from the medicament delivery device 102 directly to the cloud services/server(s) 128 or instead from the management device 104 to the cloud services/server(s) 128. The cloud services/server(s) 128 may provide output from the model 115 as needed to the management device 104 and/or medicament delivery device 102 during operation.

Other devices, like smartwatch 130, fitness monitor 132 and wearable device 134 may be part of the delivery system 100. These devices may communicate with the medicament delivery device 102 to receive information and/or issue commands to the medicament delivery device 102. These devices 130, 132 and 134 may execute computer programming instructions to perform some of the control functions otherwise performed by controller 110 or processor 119. These devices 130, 132 and 134 may include displays for displaying information. The display may show a user interface for providing input by the user, such as to request a change or pause in dosage or to request, initiate, or confirm delivery of a bolus of a medicament, or for displaying output, such as a change in dosage (e.g., of a basal delivery amount) as determined by controller 110 or management device 104. These devices 130, 132 and 134 may also have wireless communication connections with the sensor 106 to directly receive analyte measurement data.

A wide variety of medicaments may be delivered by the medicament delivery device 102. The medicament may be insulin for treating diabetes. The medicament may be glucagon for raising a user's blood glucose level. The medicament may also be a glucagon-like peptide (GLP)-1 receptor agonists for lowering blood glucose or slowing gastric emptying, thereby delaying spikes in blood glucose after a meal.

FIG. 1B shows a block diagram 138 of examples of some sensors 106 that may be used in exemplary embodiments. The sensors 106 may gather information from the user 108 that help the model 115 to determine the current phase of the menstrual cycle of the user 108 and to help adapt to changing insulin sensitivity of the user over the phases of the menstrual cycle. The sensors 106 may include a skin temperature sensor 141 for sensing the skin temperature of the user at a suitable location, such as the wrist of the user or in proximity to the location of the medicament delivery device 102. The sensors 106 may include a heart rate monitor 142 for sensing the heart rate of the user 108. A skin conductance sensor 143 may be provided to measure the skin conductance of the user 108. An activity detector 144, such as an accelerometer, may be used to measure the activity level of the user. A glucose sensor 145, such as a continuous glucose monitor (CGM), may be provided. The glucose sensor 145 may be an on-body sensor that detects the blood glucose concentration of the user at regular intervals, such as every 5 minutes.

FIG. 1C depicts one exemplary configuration of the sensors 106 relative to the medicament delivery device 102. As shown in FIG. 1C, in some exemplary embodiments, the medicament delivery device 102 includes a delivery device housing or housings 150 in which components of the medicament delivery device, like the controller 110, the reservoir 112, the pump 113 and the storage are contained. An accelerometer 160 may be contained inside the delivery device housing(s) 150. The accelerometer 160 acts as an activity detector 144 (FIG. 1B) that may be used to determine if the user is sleeping, exercising, etc. The accelerometer 160 may also identify a magnitude of activity level of the user 108.

The delivery device housing(s) 150 have an adhesive pad 152 attached to the bottom surface of the delivery device housing(s) 150. The adhesive pad 152 is secured to the delivery device housing(s) 150 by a suitable means such as by heat welding, an adhesive or other means. The adhesive pad 152 has an adhesive on its underside. The adhesive secures the adhesive pad 152 to the skin surface of the user 108. Certain sensors may be secured to the underside of the adhesive pad as shown. For example, a heart rate monitor 154, a skin temperature sensor 156, and a skin conductance sensor 158 may be secured to the underside of the adhesive pad and may contact the skin surface of the user 108.

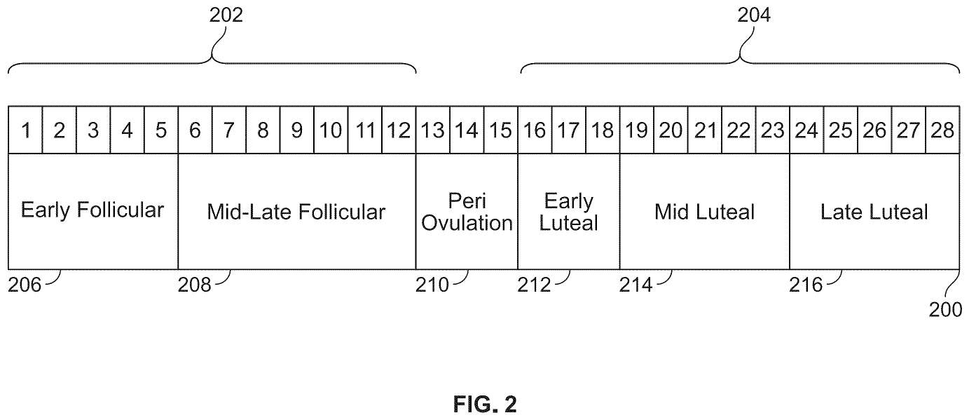

The exemplary embodiments may determine the current phase of the menstrual cycle of the user 108 and based on that determination may adjust the magnitude of the basal dosages and/or bolus dosages of medicament, such as insulin, to matching changing insulin sensitivity due to the phase of the menstrual cycle. FIG. 2 depicts a breakdown of the phases of an idealized menstrual cycle 200. The depiction begins at the time that the user begins her period. The menstrual cycle 200 includes a follicular phase 202 that precedes ovulation and a luteal phase 202 that follows ovulation. Ovulation occurs during the peri-ovulation phase 210. The follicular phase 202 contains an early follicular phase 206 and a late follicular phase 208. The luteal phase 204 contains the early luteal phase 212, the mid luteal phase 214 and the late luteal phase 216. The days are numbered from 1 to 28 along the top of the phases in FIG. 2. The duration of these phases, and hence the total duration of the cycle, may vary among users.

The insulin needs of users change over the phases of the menstrual cycle 200. Changes in estradiol and progesterone levels over the menstrual cycle 200 are correlated with changing insulin sensitivity. Insulin sensitivity is higher in the early follicular phase 206 as opposed to the luteal phase 204. The insulin needs of a user may vary by 25% within the menstrual cycle. The variations in insulin needs are not consistent for users. A customized approach is helpful. The exemplary embodiments provide such as customized approach.

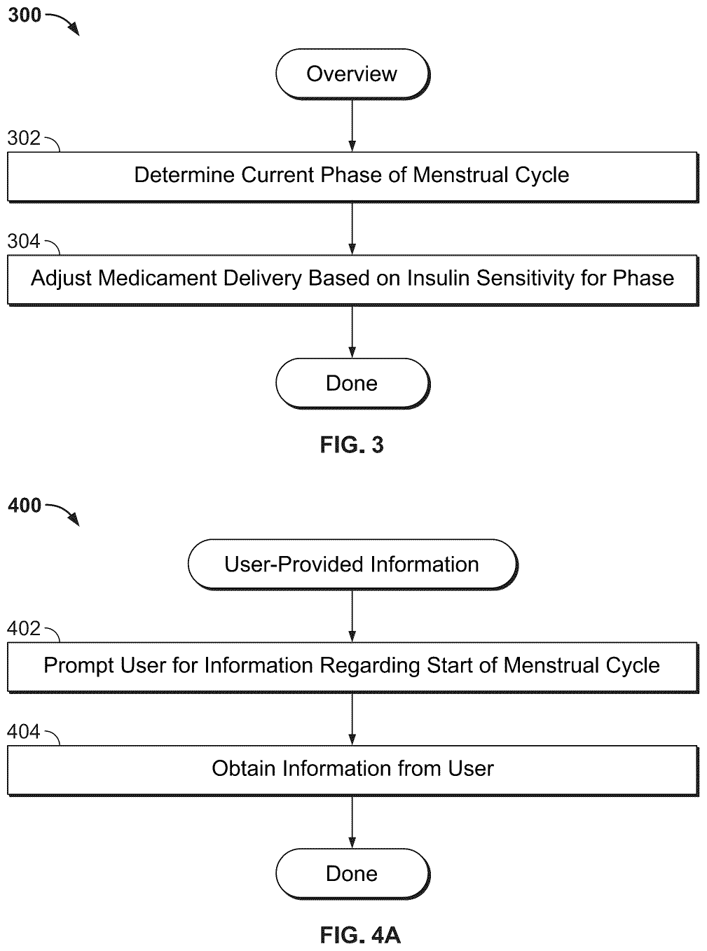

FIG. 3 depicts a flowchart 300 of basic steps that may be performed in exemplary embodiments to compensate for changing insulin needs over the course of a menstrual cycle of a user 108. First, the current phase of the menstrual cycle of the user 108 is determined at 302. This may entail obtaining data from sensors connected to the user and processing the data to determine the current phase. Alternatively, this may entail simply prompting the user 108 to provide information from which the current phase may be derived. Once the current phase is known, the medicament delivery is adjusted based on the current phase at 304.

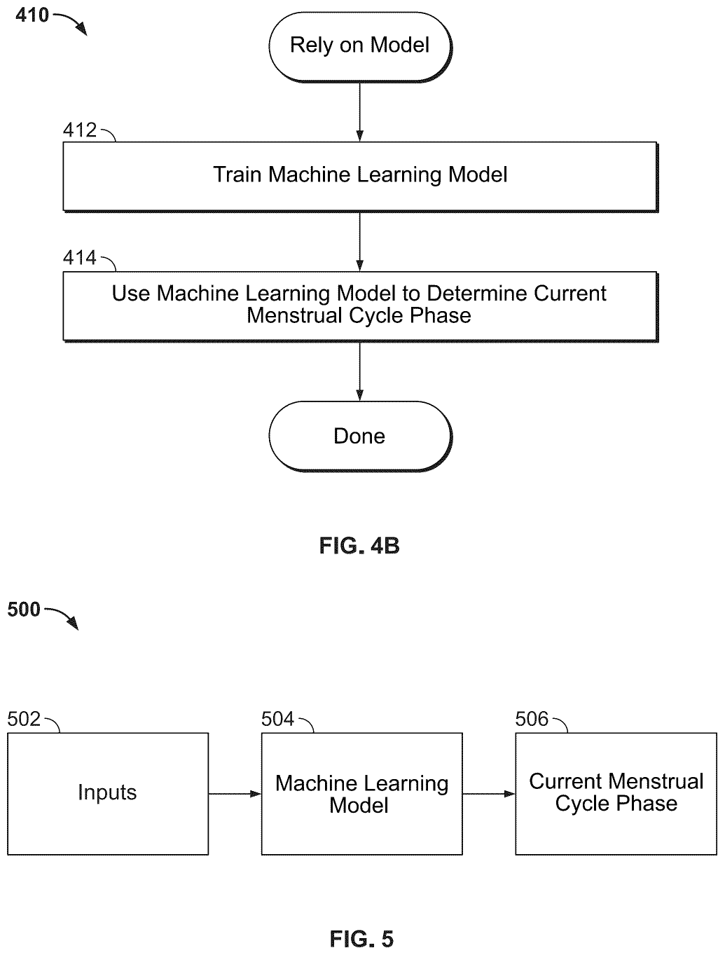

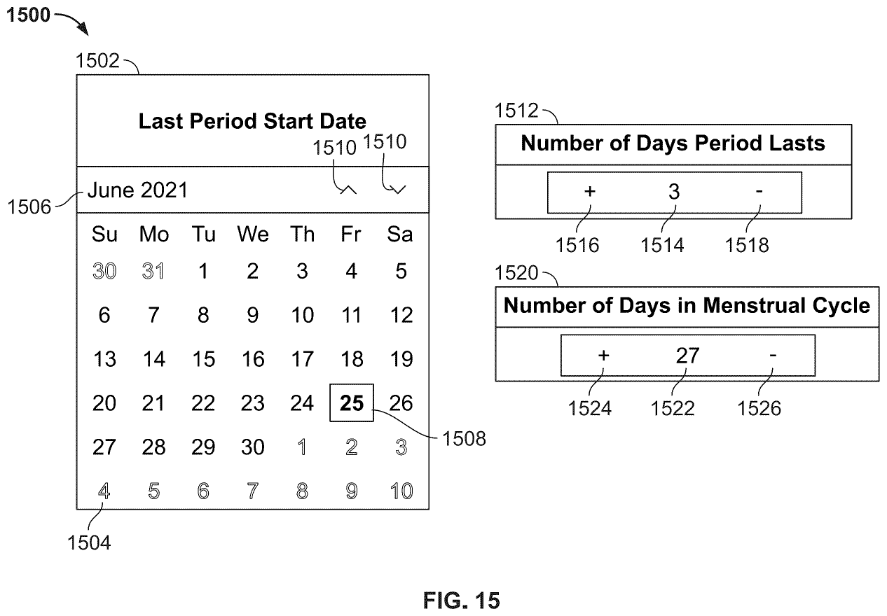

As shown in the flowchart 400 of FIG. 4A, the determination of the current phase of the menstrual cycle of the user 108 may be determined based on user provided information. At 402, the user 108 may be prompted to provide information regarding the start of the latest menstrual cycle. This may involve, for example, asking the user 108 to identify the date that their most recent period began. A prompt may be provided on the user interface 123 of the management device 104 or the user interface 117 of the medicament delivery device 102. The user might, for instance, be asked to choose a day on a monthly calendar (e.g., FIG. 15). At 404, the information is obtained from the user. The obtained information identifies the date of the beginning of the menstrual cycle of the user and may be used to identify the current phase of the menstrual cycle based on the offset of the current date from the date that the menstrual cycle began.

Another option to determining the current phase of the menstrual cycle of the user 108 is to rely upon a model 115. FIG. 4B depicts a flowchart 410 of illustrative steps that may be performed in exemplary embodiments when relying upon a model. The model 115 may be a deep learning model or other type of neural network model. The model 115 may use machine learning algorithms and may use logistic regressors, random forests, support vector machines boosting and bagging approaches and/or unsupervised learning methods. The model 115 automatically detects a current phase of the menstrual cycle of a user and learns trends in insulin sensitivity variation for the user. At 412, the machine learning model 115 is trained. The machine learning model 115 may be trained on data obtained from other women and then may be customized to the user 108. Alternatively, the machine learning model 115 may be trained solely on data for the user 108. For example, the medicament delivery device 102 may be secured to the user 108 and the sensors 106 may gather data for a period of two to three months. During this time, the machine learning model 115 may be in training mode. Once the training is complete, the machine learning model 115 is used to determine the current menstrual cycle phase of the user 108 at 414.

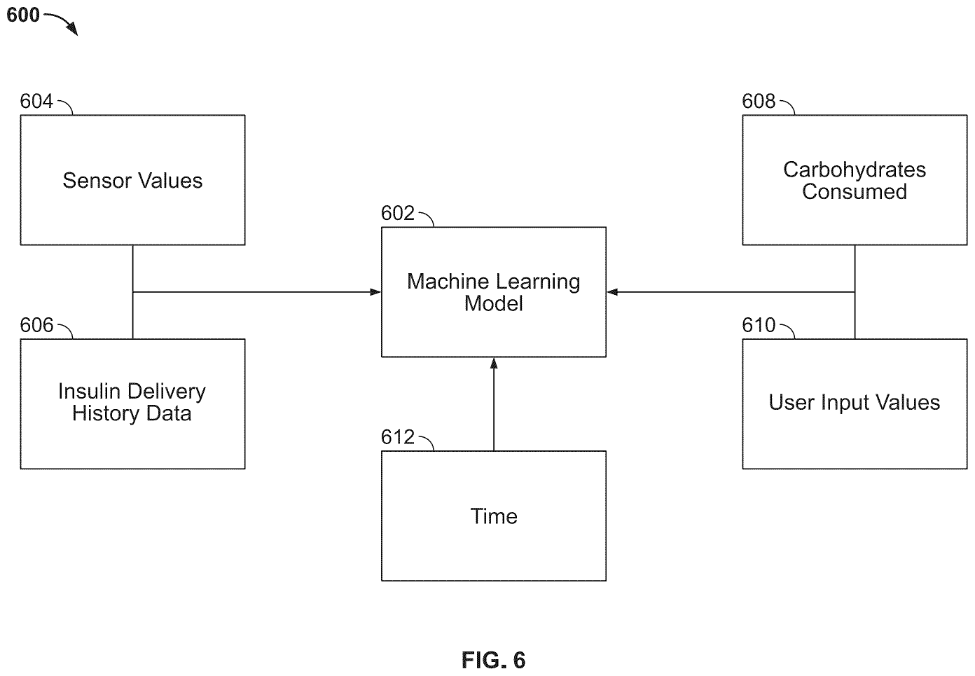

FIG. 5 depicts a block diagram 500 of the data flow for the machine learning model 504. The machine learning model 504 processes inputs 502 and generates an output 506 of the current menstrual cycle phase. FIG. 6 depicts a diagram 600 showing some of the inputs that may be used by the machine learning model 602 in exemplary embodiments. Many of these inputs are sensor values 604. For example, the sensor values may include values from sensors like those shown in FIG. 1B of a skin temperature sensor 141, a heart rate sensor 142, a skin conductance sensor 143, an activity detector 144 and/or a glucose sensor 145. The sensor values 604 are segregated into nighttime readings (as indicated by time values when taken) and daytime readings where the user is at rest (as determined by the accelerometer readings or other activity level sensor readings). These values are more determinative of a current phase of menstrual cycle of the user 108 than values taken when the user 108 is active. The machine learning model 602 may use insulin delivery histories 606 as input. Carbohydrates consumed 608 may be input as well as user input values 610. Carbohydrate consumption levels may be indicative of a current phase of the menstrual cycle of the user 108. The machine learning model 602 may also receive time and/or date values 612 as input.

Skin temperature generally drops in the follicular phase 202 and for most women is significantly higher in the luteal phase 204. Nightly heart rate may be lower than average in the follicular phase 202 and higher than average in the luteal phase 204. Nightly heart rate variability may be higher in the follicular phase 202 than average and lower than average in the luteal phase 204. Skin conductance may be lower in the follicular phase 202 than in other portions of the menstrual cycle. Increased carbohydrates are typically consumed during the luteal phase 204. These observations may be used by the logic or the machine learning model 602 to determine current phase of the menstrual cycle of the user.

The machine learning model 602 may determine whether the current phase of the user is the follicular phase 204 by performing the steps of the flowchart 700 in exemplary embodiments. The machine learning model 602 may be rules based in some exemplary embodiments. The flowchart 700 encodes some of the logic used by a general rule of the machine learning model 602. These steps may in some exemplary embodiments be performed by software that is not part of a machine learning model.

At 702, a determination is made whether a current skin temperature of the user 108 is greater than a mean skin temperature of the user over a time interval, such as over one or more menstrual cycles. This check is made because, in general, during the follicular phase the skin temperature of a woman is elevated above the mean skin temperature. If not, the conclusion is reached that the current phase is not a follicular phase at 708. If the skin temperature is greater than the average skin temperature, the variability of the heart rate of the user 108 is compared to the mean heart rate variability of the user to determine if the heart rate variability is greater than the average heart rate variability over an interval at 704. Heart rate variability refers to the degree to which heart rate varies over a menstrual cycle and the mean heart rate variability refers to the average heart rate variability over multiple menstrual cycles, such as those for which data was gathered in the training. It is known that heart rate variability increases to be above average for many women during the follicular phase 204. If heart rate variability is not higher than the mean, it is concluded that the current phase is not a follicular phase at 708. If heart rate variability of the user is above the mean, the current heart rate of the user is compared to the mean heart rate at 706. If the current heart rate is above the mean heart rate, it is determined that the current phase is the follicular phase 204 at 710. Otherwise, the current phase is determined to not be the follicular phase at 708.

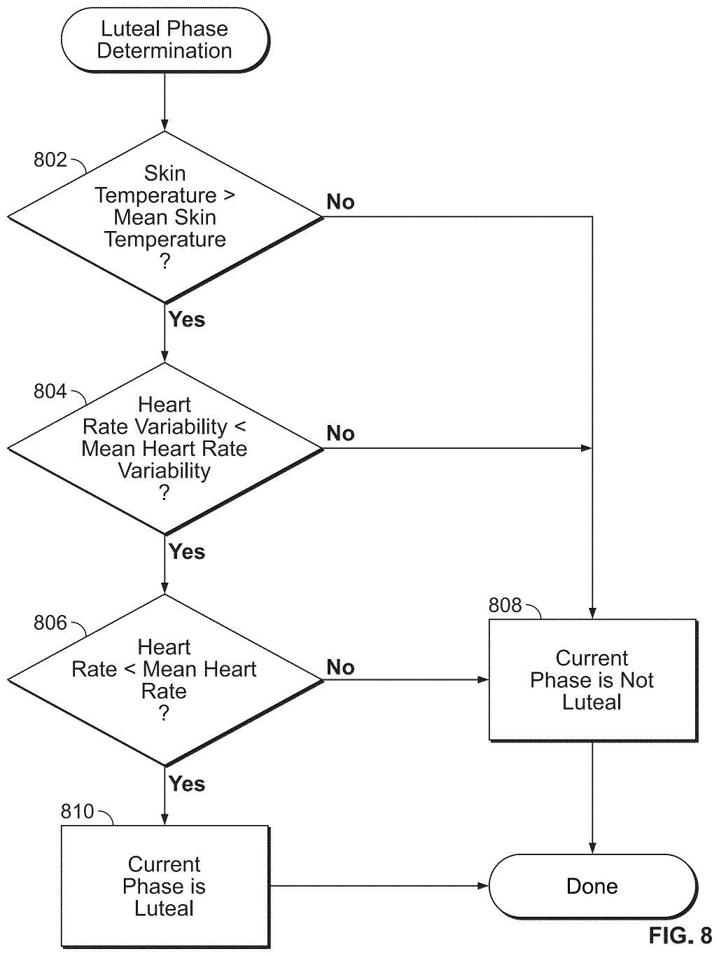

FIG. 8 depicts a flowchart 800 of logic that may be applied in exemplary embodiments by the machine learning model 602 or by logic to determine if the current phase is the luteal phase 204. In general, a number of checks are made to see if sensed values are indicative of the current phase being the luteal phase 204. The flowchart 800 captures a general rule that may be applied by the machine learning model 602. At 802, a check is made whether a current skin temperature is higher than the mean skin temperature for the user 108. If not, the current phase is determined to not be the luteal phase at 808. If so, a check is made whether the current heart rate variability of the user is greater than the mean heart rate variability at 804. If not, the current phase is determined to not be the luteal phase at 808. If so, at 806 a check is made if the heart rate of the user is less than the mean heart rate. If not, the current phase is determined to not be the luteal phase 204 at 808. If so, the current phase is determined to be the luteal phase 204 at 810.

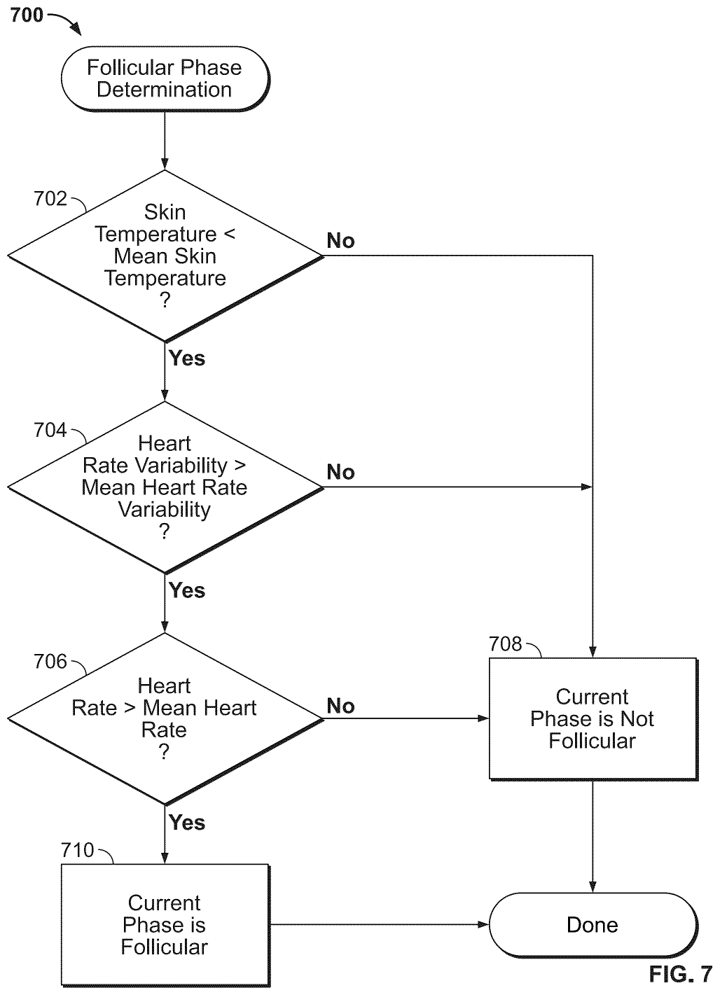

The machine learning model need not be limited to applying the general rules and logic of FIGS. 7 and 8. Detailed individual rules may also be learned and applied by the machine learning model 602.

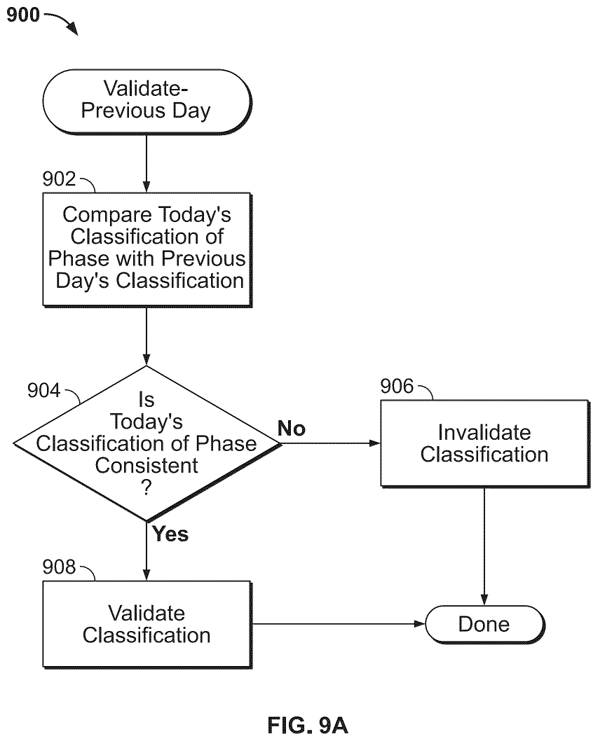

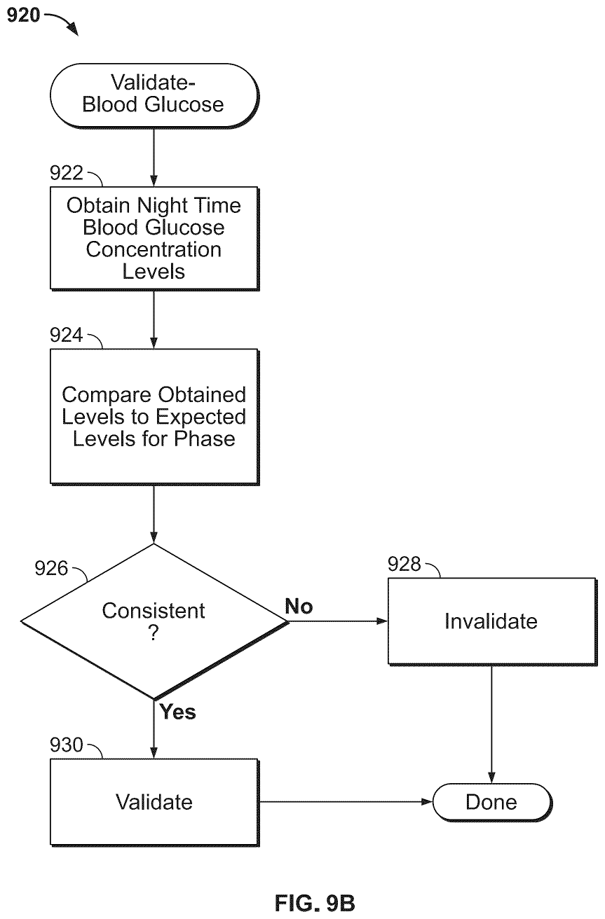

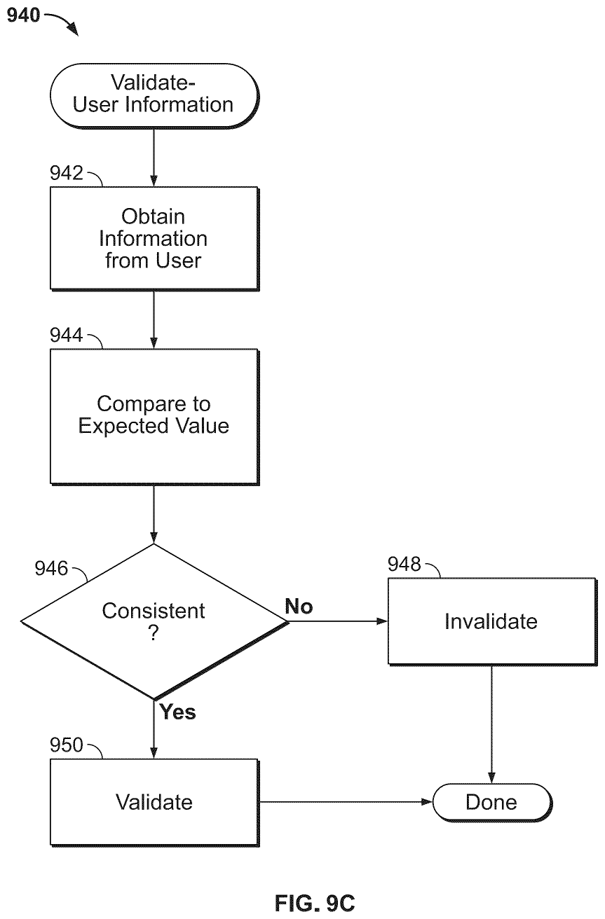

The choice of current phase by the machine learning model 602 may be validated in multiple ways. One validation option is to look at previous decisions of a current phase made by the machine learning model 602. FIG. 9A depicts a flowchart 900 of illustrative steps that may be performed by exemplary embodiments to perform such validation. At 902, the classification of phase for the previous day by the machine learning model 602 is compared with the classification of current phase chosen for the current day by the machine learning model 602. An analysis is made whether the classifications are consistent at 904. In other words, does the current day's classification of current phase make sense with yesterday's classification. For example, if yesterday's classification was the early follicular phase and today's classification was the mid luteal phase, there is an inconsistency as these phases are not adjacent to each other and do not follow each other. If there is an inconsistency, the classification of the current phase is invalidated at 906. If there is not an inconsistency, the classification of the current phase is validated at 908.

Another validation option is to look at the blood glucose concentration levels for the user to validate the classification of the current phase by the machine learning model 602. FIG. 9B depicts a flowchart 920 of illustrative steps that may be performed in exemplary embodiments to validate the classification of the current phase. At 922, the nighttime blood glucose concentration levels for the user are obtained. The blood glucose sensor 145 may provide these values. These obtained sensor values are compared to the expected values for the current phase classification from the machine learning model 602 at 926. For example, if there is a reduction in nighttime blood glucose concentration levels relative to a baseline, it is an indication that there is increased insulin sensitivity. An increase in insulin sensitivity is associated with follicular phase 202 and a decrease in insulin sensitivity is associated with the luteal phase 204. At 926, a check is made whether the nighttime blood glucose concentration levels are consistent with the classification of the current phase by the machine learning model 602. If not, the classification of the current phase is invalidated at 928. If so, the classification of the current phase is validated at 930.

A third approach to validation of a classification of the current phase by the machine learning model 602 is to validate relative to information provided by the user 108. FIG. 9C depicts a flowchart 940 of illustrative steps that may be performed in exemplary embodiments to perform such validation. At 942, information is obtained from the user 108. For instance, the date that a last period of the user started may be obtained from the user. This may be compared at 944 to an expected value, e.g., the date the machine learning model 602 believes the menstrual cycle of the user began. At 946, a check is made whether the machine learning information (e.g., date) is consistent with the information (e.g., date) provided by the user 108. If inconsistent, the current phase classification by the machine learning model 602 is invalidated at 948. If consistent, the current phase classification by the machine learning model 602 is validated at 950.

The three above-described validation approaches may be applied together, separately or in various combinations by the machine learning model 602 to validate preliminary classifications of the current phase of the menstrual cycle by the machine learning model. These validations may be performed before the classification of the current phase is used to adapt medicament dosages. The validations provide an added level of confidence that the classifications of current phase are accurate and reliable. The consequences of invalidation may be determined by the machine learning model 602. Invalidation may prompt the machine learning model 602 to repeat the classification of the current phase, for instance.

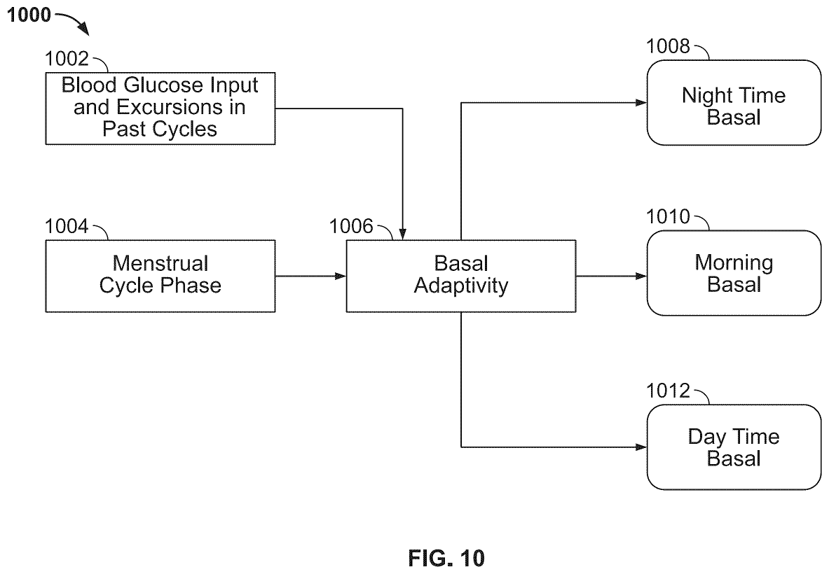

Once the current phase of the menstrual cycle of the user 108 is determined, the basal dosage of medicament to be delivered by the medicament delivery device 102 may be adjusted as needed. FIG. 10 depicts a diagram 1000 showing the inputs an output of the basal adaptivity mechanism 1006 of the exemplary embodiments. The basal adaptivity mechanism 1006 may be realized by computer programming instructions contained in the control applications 116 or 120. The basal adaptivity mechanism 1006 receives the classification of the current phase 1004 of the menstrual cycle of the user 108. The basal adaptivity mechanism 1006 also receives a most recent blood glucose concentration reading and information regarding excursions from past cycles (a cycle is, for example, a 5-minute interval). The basal adaptivity mechanism 1006 considers basal needs separately for nighttime, morning and daytime because the needs at these portions of the day tend to vary. As such, depending on the time of day, the basal adaptivity mechanism 1006 may produce a nighttime basal dosage 1008, a morning basal dosage 1010 or a daytime basal dosage 1012. In general, the basal adaptivity mechanism 1006 looks at the current menstrual phase classification 1004 and the historical blood glucose concentration trends in earlier menstrual cycles to adapt the basal dosage amounts 1008, 1010 and 1012. In general, the dosages of basal insulin are increased in the early luteal phase 212 and gradually return to normal levels at the end of the luteal phase 204. The adaptation is tailored to the individual user based on learned patterns. Past blood glucose concentration excursions 1002 are referenced in determining customized basal dosages.

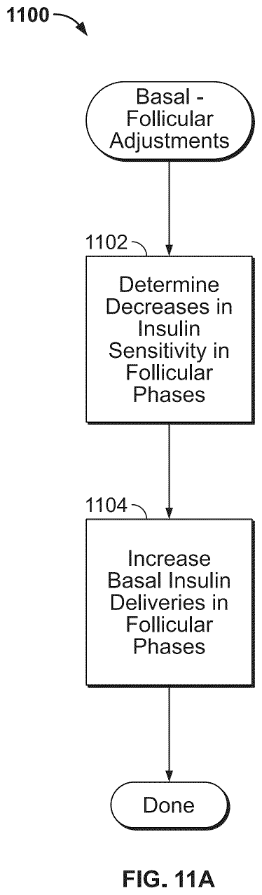

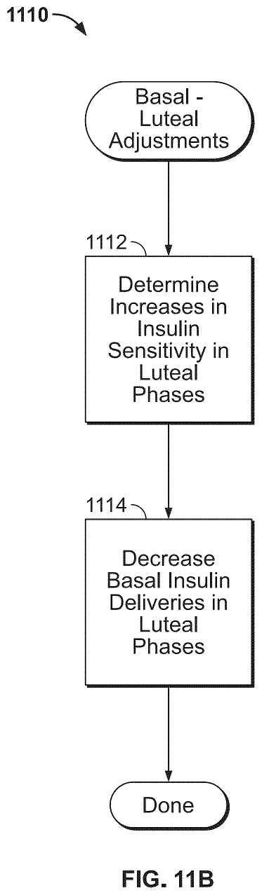

FIG. 11A depicts a flowchart 1100 of illustrative steps that may be performed in exemplary embodiments for follicular phase 202 adjustments. As noted above, decreases in insulin sensitivity are observed with some women in the follicular phase 202. At 1102, the decreases in insulin sensitivity are determined. As mentioned above these decreases are determined by observation by the machine learning model 602. The basal insulin dosages are increased in the follicular phases to compensate for the decreased insulin sensitivity at 1104.

FIG. 11B depicts a flowchart 1110 of illustrative steps that may be performed in exemplary embodiments for luteal phase 204 adjustments. At 1112, a determination of increases in insulin sensitivity in the luteal phase 204 is determined. The basal insulin dosages for the luteal phase 204 are decreased in response at 1114.

Some examples help to illustrate the basal dosage adaptivity that may be provided. The adaptivity is customized to the particular user. Some women experience a reduction in insulin sensitivity of up to 25% in the early luteal phase 212. Typical reductions of insulin sensitivity are in excess of 5%. In such instances, a nominal basal dosage would be set for the early follicular phase 206, the mid-late follicular phase 208 and the peri-ovulation phase 210. The basal dosage would be increased by 15% during the early luteal phase 212. The increase relative to the nominal basal dosage would be set at 10% in the mid luteal phase 214 and would be set at a 5% increase relative to the nominal basal dosage in the late luteal phase 216.

Some women may experience an increased insulin sensitivity in the luteal phases 212, 214 and 216. The increases may be 5% in the early luteal phase, and 10% in the mid luteal phase 214. For these women, there is a nominal basal dosage for the follicular phase 202, the peri-ovulation phase 210 and the late luteal phase 216. The basal dosage would be decreased by 5% relative to the nominal basal dosage in the early luteal phase 212 and would be decreased by 10% relative to the nominal basal dosage in the mid luteal phase 214.

In some instances, no significant changes in insulin dosages for users are seen. The basal dosages are not adapted for such users.

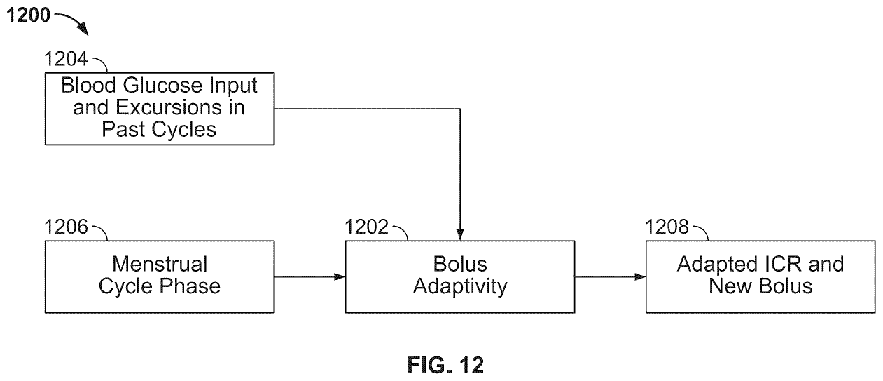

The bolus dosages may also be adapted. FIG. 12 depicts a diagram 120 showing illustrative inputs and outputs for a bolus adaptivity mechanism 1202. The bolus adaptivity mechanism 1202 may be realized as computer programming instructions in the control application 116. Inputs to the bolus adaptivity mechanism 1202 may include the classification of the current menstrual cycle phase 1206 and the current blood glucose concentration reading along with information regarding blood glucose concentration excursions from past cycles 1204. The bolus adaptivity mechanism 1202 processes these inputs 1204 and 1206 to produce an adapted insulin to carbohydrate ratio for the user and a bolus dosage amount 1208. Bolus dosages may be calculated when requested by the user 108.

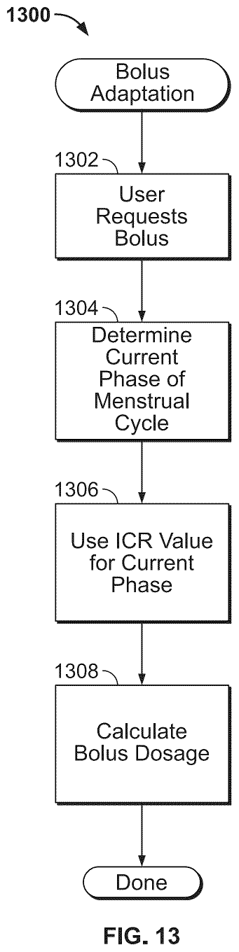

FIG. 13 depicts a flowchart 1300 of illustrative steps that may be performed in exemplary embodiments to generate an adapted insulin bolus dosage based on the current phase of the menstrual cycle of the user 108. At 1302, the user 108 requests a medicament bolus, such as a bolus of insulin. At 1304, the current phase of the menstrual cycle of the user 108 is determined as discussed above. As was mentioned above, insulin sensitivity may vary with phase of the menstrual cycle. The exemplary embodiments may adapt for the changing insulin sensitivity by adapting the insulin to carbohydrates ratio (ICR), which may be used in determining the bolus dosage. The ICR is used to determine how much insulin is needed to compensate for a quantity of carbohydrates that is ingested. If insulin sensitivity increases, the ICR is decreased, whereas if insulin sensitivity decreases, the ICR is increased. At 1306, the ICR for the current phase of the menstrual cycle of the user 108 is identified and at 1308 is used to calculate the bolus dosage. The magnitude of the increase in ICR or decrease in ICR is customized to the user's learned change in insulin sensitivity across the phases of the menstrual cycle of the user.

The bolus dosage is proportional to the carbohydrates ingested and the ICR. In particular, the bolus dosage equals the grams of carbohydrates ingested divided by the ICR. ICR typically will be higher during the luteal phase 204 compared to the follicular phase 202. A suitable increase in the ICR for the luteal phase is 10% compared to the follicular phase 202.

The ICR adaptation may follow the insulin sensitivity trend. As explained above, the insulin sensitivity changes at the individual level. As an example, suppose that the insulin sensitivity decreases in the early luteal phase 212 as described above, sensitivity changes the ICR would be reduced by 15% compared to baseline ICR in the early luteal phase 212, increased by 10% in the mid luteal phase 214 and reduced by 5% in the late luteal phase 216 compared to baseline ICR.

When the insulin sensitivity does not change through the menstrual cycle, the ICR may not be modulated through the menstrual period. The insulin sensitivity may be determined, such as described above, by looking at changes in blood glucose concentration for the user relative to the quantity of insulin delivered. If the area under the blood glucose curve is remaining the same, but the insulin delivery amount is increased, this means that the insulin sensitivity has been reduced (for the same blood sugar levels we are requiring more insulin). Conversely, if the area under the blood glucose curve remains the same, but the amount of insulin needed has reduced, then the insulin sensitivity has increased. When comparing insulin sensitivity across the menstrual cycle one can compare the ratio of blood sugar area under the curve to insulin delivery and infer insulin sensitivity variation or the lack thereof. If the change in insulin sensitivity over the user's menstrual cycle is below a threshold based on the comparison, the modulation by menstrual cycle phase described herein may not be performed.

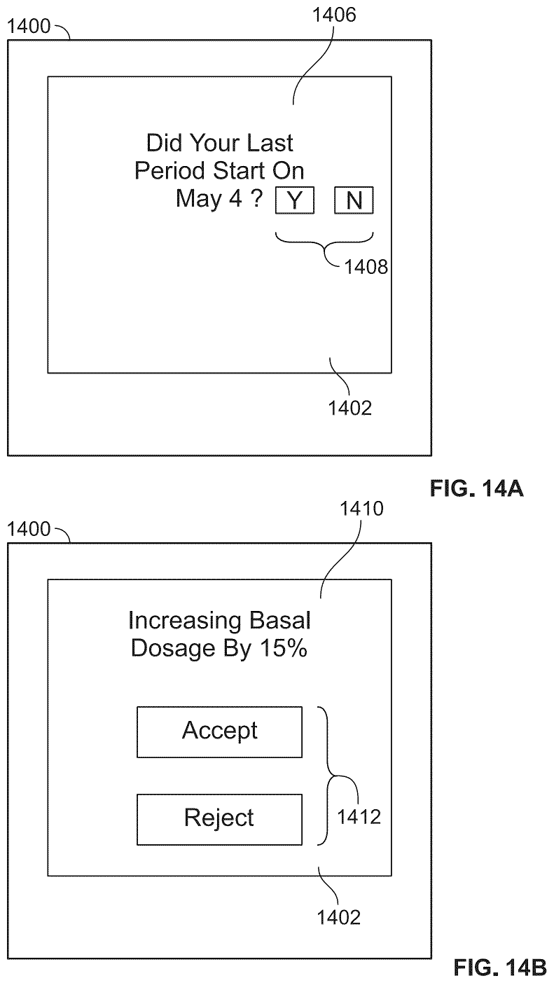

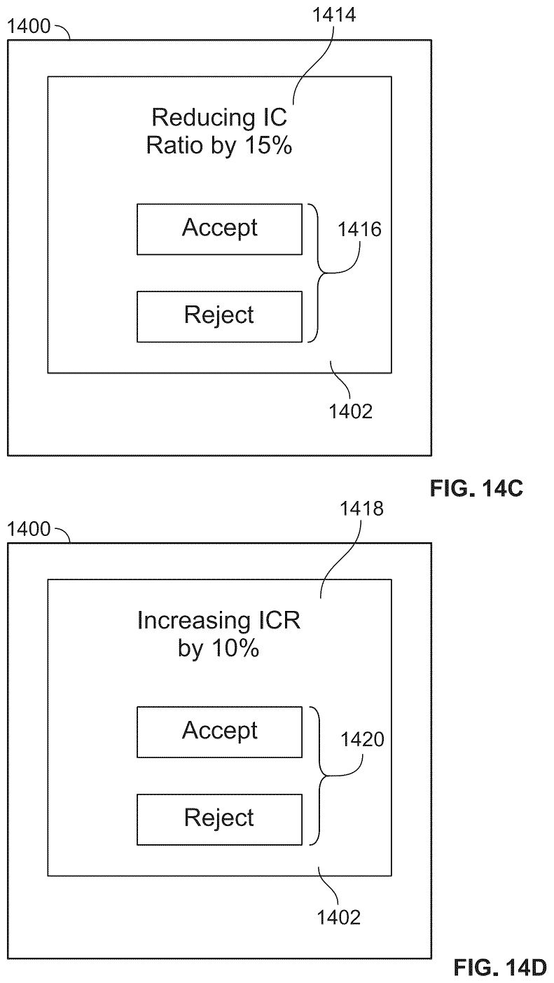

As was mentioned above, the medicament delivery device 102 may include a user interface 117 and the management device 104 may include a user interface 123. The user interfaces 117 and 123 may be used to verify the phase that the machine learning model deems to be the current phase of the menstrual cycle. For example, as shown in FIG. 14A, a display 1402 on a device 1400 (such as the medicament delivery device 102 or the management deice 104) may display a prompt 1406 for the user to verify and/or input the date that her last period began on. Buttons 1408 may be selected to verify (selecting “Y”) or not verify (selecting “N”) the displayed date. Buttons may be used to adjust the date rather than selecting a “Y” or “N” to verify the date. The display 1402 may also be used to verify a change in the basal dosage. As shown in FIG. 14B, a message 1410 that may be displayed on display 1402 to prompt the user to accept or reject the proposed increase in basal dosage. A user interface screen or informational text may also be displayed to inform the user as to why an increase or decrease of X % (e.g., 10%) of the basal dosage is being recommended by the system. Buttons 1412 enable the user 108 to accept or reject the proposed increase. The display 1402 may also display a request 1414 to accept or reject a proposed reduction in ICR by choosing buttons 1416 as shown in FIG. 14C. A user interface screen or informational text may also be displayed to inform the user as to why an increase or decrease of the ICR (e.g., −15%) is being recommended by the system. Similarly, a request 1418 to reduce or increase the ICR may be displayed on the display 1402. Buttons 1420 enable the user to accept or reject the reduction. Such buttons, confirmations, recommendations, and/or informational text may be displayed and/or used throughout the menstrual cycle.

These are just a few examples of how the user interface 117 or 123 may be used to authorize changes, inform of changes, recommend changes, or confirm analysis performed by the machine learning model 602. Other messages and graphical items may be displayed.

FIG. 15 shows an illustrative user interface 1500 for soliciting information from the user 108. The user interface includes a section 1502 for obtaining information regarding the start date of the user's last period. The section 1502 includes a calendar 1504 that shows a month. Text 1506 identifies the month and year that is displayed. The current date is identified by a highlight box 1508. An up arrow and a down arrow 1510 may be selected to choose the next month or the previous month, respectively. The user 108 simply clicks on the day of the month on which her last period started. This information is then recorded and stored, such as in storage 114. The user interface 1500 may also include a text box 1512 for the user 108 to specify the number of days her period typically lasts. The user 108 may enter a value 1514 in the text box 1512 or may be select the + button 1516 to increase the displayed value 1514 or the − button to decrease the displayed value by increments of 1. A text box 1520 may be provided for the user 108 to specify the average number of days in her menstrual cycle. The user 108 may enter a value 1522 in the text box 1520 or use the + button 1524 and/or − button to select the correct value. The information gathered from the user may be stored in storage 114. This information may be used by the model 155 and the controller 110 as described above.

While the discussion has focused on exemplary embodiments, it should be appreciated that various changes in form and detail relative to the exemplary embodiments without departing from the intended scope of the appended claims.

1. A medicament delivery system, comprising:
at least one sensor chosen from the group of a skin temperature sensor, a heart rate sensor, a skin conductance sensor, or an activity detector;
a storage for storing computer programming instructions; and
a processor configured to execute the computer programming instructions, wherein executing the computer programming instructions causes the processor to:
receive sensor output for a user from the at least one sensor;
process the sensor output to determine a current phase of a menstrual cycle of the user;
determine a dose of medicament to be delivered to the user based at least in part on the determined current phase of the menstrual cycle of the user.

2. The medicament delivery system of claim 1, wherein the at least one sensor comprises multiple sensors.

3. The medicament delivery system of claim 2, wherein the multiple sensors include the heart rate sensor and the skin temperature sensor.

4. The medicament delivery system of claim 1, wherein the medicament delivery system includes an adhesive pad and wherein the at least one sensor is secured to the adhesive pad.

5. The medicament delivery system of claim 1, wherein the at least one sensor includes the heart rate sensor and wherein the processing of the sensor output comprises analyzing at least one of heart rate variability or heart rate.

6. The medicament delivery system of claim 1, wherein the at least one sensor includes the activity sensor and wherein the activity sensor is an accelerometer.

7. The medicament delivery system of claim 1, wherein the determining a dose of medicament comprises determining an insulin sensitivity of the user based on the determined current phase of the menstrual cycle of the user and determining the dose of medicament in view of the determined insulin sensitivity.

8. A medicament delivery system, comprising:
a medicament storage for storing a medicament;
a pump for pumping the medicament from the medicament storage;
a storage medium for storing computer programming instructions; and
a processor configured for executing the computer programming instructions to cause the processor to:
compare most recent skin temperature to a mean skin temperature of the user over a time interval;
where the most recent skin temperature of the user is not greater than the mean skin temperature of the user over the time interval, determine that the user is not in a luteal phase of their menstrual cycle;
where the most recent skin temperature of the user is greater than the mean skin temperature of the user over the time interval, analyze heart rate data to determine if the user is in the luteal phase of their menstrual cycle.

9. The medicament delivery system of claim 8, wherein the analyzing of the heart rate data comprises comparing a current heart rate of the user with a mean heart rate of the user for the time interval.

10. The medicament delivery system of claim 9, wherein executing the computer programming instructions further comprises determining that the user is not in the luteal phase of their menstrual cycle where the current heart rate of the user is not less than the mean heart rate of the user for the time interval.

11. The medicament delivery system of claim 8, wherein the analyzing of the heart rate data comprises comparing a current magnitude of heart rate variability of the user with a mean magnitude of heart rate variability of the user for the time interval.

12. The medicament delivery system of claim 11, wherein executing the computer programming instructions further comprises determining that the user is not in the luteal phase of their menstrual cycle where the current magnitude of heart rate variability of the user is not less than the mean magnitude of heart rate variability for the time interval.

13. The medicament delivery system of claim 8, the analyzing of the heart rate data to determine if the user is in the luteal phase of their menstrual cycle comprises determining that the user is in the luteal phase of their menstrual cycle where a current heart rate of the user is less than a mean heart rate of the user for the time interval and a current magnitude of heart rate variability of the user is less than a mean magnitude of heart rate variability of the user for the time interval.

14. A medicament delivery system, comprising:
a medicament storage for storing a medicament;
a pump for pumping the medicament from the medicament storage;
a storage medium for storing computer programming instructions; and
a processor configured for executing the computer programming instructions to cause the processor to:
compare most recent skin temperature to a mean skin temperature of the user over a time interval;
where the most recent skin temperature of the user is not less than the mean skin temperature of the user over the time interval, determine that the user is not in a follicular phase of their menstrual cycle;
where the most recent skin temperature of the user is not less than the mean skin temperature of the user over the time interval, analyze heart rate data to determine if the user is in the luteal phase of their menstrual cycle.

15. The medicament delivery system of claim 14, wherein the analyzing of the heart rate data comprises comparing a current heart rate of the user with a mean heart rate of the user for the time interval.

16. The medicament delivery system of claim 15, wherein executing the computer programming instructions further comprises determining that the user is not in the follicular phase of their menstrual cycle where the current heart rate of the user is not greater than the mean heart rate of the user for the time interval.

17. The medicament delivery system of claim 14, wherein the analyzing of the heart rate data comprises comparing a current magnitude of heart rate variability of the user with a mean magnitude of heart rate variability of the user for the time interval.

18. The medicament delivery system of claim 17, wherein executing the computer programming instructions further comprises determining that the user is not in the follicular phase of their menstrual cycle where the current magnitude of heart rate variability of the user is not greater than the mean magnitude of heart rate variability for the time interval.

19. The medicament delivery system of claim 14, the analyzing of the heart rate data to determine if the user is in the follicular phase of their menstrual cycle comprises determining that the user is in the follicular phase of their menstrual cycle where a current heart rate of the user is greater than a mean heart rate of the user for the time interval and a current magnitude of heart rate variability of the user is greater than a mean magnitude of heart rate variability of the user for the time interval.

20. The medicament delivery device of claim 14, wherein the medicament comprises insulin.
